SEQUENCE LISTING (91 sequences)

SEQ ID NO: 1 - protein, 17 aa, Melanoma Associated Endogenous Retrovirus
EMQRKAPPRRRRHRNRA

SEQ ID NO: 2 - protein, 17 aa, Melanoma Associated Endogenous Retrovirus
RMKLPSTKKAEPPTWAQ

SEQ ID NO: 3 - protein, 17 aa, Melanoma Associated Endogenous Retrovirus
TKKAEPPTWAQLKKLTQ

SEQ ID NO: 4 - protein, 17 aa, Melanoma Associated Endogenous Retrovirus
MPAGAAAANYTYWAYVP

SEQ ID NO: 5 - protein, 17 aa, Melanoma Associated Endogenous Retrovirus
PIDDRCPAKPEEEGMMI

SEQ ID NO: 6 - protein, 17 aa, Melanoma Associated Endogenous Retrovirus
YPPICLGRAPGCLMPAV

SEQ ID NO: 7 - protein, 17 aa, Melanoma Associated Endogenous Retrovirus
YQRSLKFRPKGKPCPKE

SEQ ID NO: 8 - protein, 17 aa, Melanoma Associated Endogenous Retrovirus
FRPKGKPCPKEIPKESK

SEQ ID NO: 9 - protein, 17 aa, Melanoma Associated Endogenous Retrovirus
GKPCPKEIPKESKNTEV

SEQ ID NO: 10 - protein, 17 aa, Melanoma Associated Endogenous Retrovirus
GTIIDWAPRGQFYHNCS

SEQ ID NO: 11 - protein, 17 aa, Melanoma Associated Endogenous Retrovirus
RGQFYHNCSGQTQSCPS

SEQ ID NO: 12 - protein, 17 aa, Melanoma Associated Endogenous Retrovirus
DLTESLDKHKHKKLQSF

SEQ ID NO: 13 - protein, 17 aa, Melanoma Associated Endogenous Retrovirus
PWGWGEKGISTPRPKIV

SEQ ID NO: 14 - protein, 17 aa, Melanoma Associated Endogenous Retrovirus
PKIVSPVSGPEHPELWR

SEQ ID NO: 15 - protein, 17 aa, Melanoma Associated Endogenous Retrovirus
CPWFPEQGTLDLKDWKR

SEQ ID NO: 16 - protein, 17 aa, Melanoma Associated Endogenous Retrovirus
IGKELKQAGRKGNIIPL

SEQ ID NO: 17 - protein, 17 aa, Melanoma Associated Endogenous Retrovirus
DCNENTRKKSQKETEGL

SEQ ID NO: 18 - protein, 17 aa, Melanoma Associated Endogenous Retrovirus
TLKLEGKGPELVGPSES

SEQ ID NO: 19 - protein, 17 aa, Melanoma Associated Endogenous Retrovirus
GPSESKPRGTSPLPAGQ

SEQ ID NO: 20 - protein, 17 aa, Melanoma Associated Endogenous Retrovirus
QPQTQVKENKTQPPVAY

SEQ ID NO: 21 - protein, 17 aa, Melanoma Associated Endogenous Retrovirus
PAELQYRPPPESQYGYP

SEQ ID NO: 22 - protein, 17 aa, Melanoma Associated Endogenous Retrovirus
MPPAPQGRAPYPQPPTR

SEQ ID NO: 23 - protein, 17 aa, Melanoma Associated Endogenous Retrovirus
EIIDKSRKEGDTEAWQF

SEQ ID NO: 24 - protein, 17 aa, Melanoma Associated Endogenous Retrovirus
MPPGEGAQEGEPPTVEA

SEQ ID NO: 25 - protein, 17 aa, Melanoma Associated Endogenous Retrovirus
MKEGVKQYGPNSPYMRT

SEQ ID NO: 26 - protein, 17 aa, Melanoma Associated Endogenous Retrovirus
VQEQVQRNRAANPPVNI

SEQ ID NO: 27 - protein, 17 aa, Melanoma Associated Endogenous Retrovirus
LRAWEKIQDPGSTCPSF

SEQ ID NO: 28 - protein, 17 aa, Melanoma Associated Endogenous Retrovirus
TVRQSSKEPYPDFVARL

SEQ ID NO: 29 - protein, 17 aa, Melanoma Associated Endogenous Retrovirus
QSAIKPLKGKVPAGSDV

SEQ ID NO: 30 - protein, 17 aa, Melanoma Associated Endogenous Retrovirus
TGREPPDLCPRCKKGKH

SEQ ID NO: 31 - protein, 17 aa, Melanoma Associated Endogenous Retrovirus
LSGNEQRGQPQAPQQTG

SEQ ID NO: 32 - protein, 17 aa, Melanoma Associated Endogenous Retrovirus
QPFVPQGFQGQQPPLSQ

SEQ ID NO: 33 - protein, 17 aa, Melanoma Associated Endogenous Retrovirus
QLPQYNNCPPPQAAVQQ

SEQ ID NO: 34 - protein, 17 aa, Melanoma Associated Endogenous Retrovirus
AINNKEPATRFQWKVLP

SEQ ID NO: 35 - protein, 17 aa, Melanoma Associated Endogenous Retrovirus
ENRKIKPQKIEIRKDTL

SEQ ID NO: 36 - protein, 17 aa, Melanoma Associated Endogenous Retrovirus
ILPKITRREPLENALTV

SEQ ID NO: 37 - protein, 17 aa, Melanoma Associated Endogenous Retrovirus
FTDGSSNGKAAYTGPKE

SEQ ID NO: 38 - protein, 17 aa, Melanoma Associated Endogenous Retrovirus
PKERVIKTPYQSAQRAE

SEQ ID NO: 39 - protein, 17 aa, Melanoma Associated Endogenous Retrovirus
LPGPLTKANEEADLLVS

SEQ ID NO: 40 - protein, 17 aa, Melanoma Associated Endogenous Retrovirus
LKNKFDVTWKQAKDIVQ

SEQ ID NO: 41 - protein, 17 aa, Melanoma Associated Endogenous Retrovirus
PTQEAGVNPRGLCPNAL

SEQ ID NO: 42 - protein, 17 aa, Melanoma Associated Endogenous Retrovirus
IWATCQTGESTSHVKKH

SEQ ID NO: 43 - protein, 17 aa, Melanoma Associated Endogenous Retrovirus
VPEKIKTDNGPGYCSKA

SEQ ID NO: 44 - protein, 17 aa, Melanoma Associated Endogenous Retrovirus
LVKQKEGGDSKECTTPQ

SEQ ID NO: 45 - protein, 17 aa, Melanoma Associated Endogenous Retrovirus
AEQHLTGKKNSPHEGKL

SEQ ID NO: 46 - protein, 17 aa, Melanoma Associated Endogenous Retrovirus
IWWKDNKNKTWEIGKVI

SEQ ID NO: 47 - protein, 17 aa, Melanoma Associated Endogenous Retrovirus
PRVNYLQDFSYQRSLKF

SEQ ID NO: 48 - protein, 17 aa, Melanoma Associated Endogenous Retrovirus
RVNYLQDFSYQRSLKFR

SEQ ID NO: 49 - protein, 17 aa, Melanoma Associated Endogenous Retrovirus
VNYLQDFSYQRSLKFRP

SEQ ID NO: 50 - protein, 17 aa, Melanoma Associated Endogenous Retrovirus
NYLQDFSYQRSLKFRPK

SEQ ID NO: 51 - protein, 17 aa, Melanoma Associated Endogenous Retrovirus
YLQDFSYQRSLKFRPKG

SEQ ID NO: 52 - protein, 17 aa, Melanoma Associated Endogenous Retrovirus
LQDFSYQRSLKFRPKGK

SEQ ID NO: 53 - protein, 17 aa, Melanoma Associated Endogenous Retrovirus
QDFSYQRSLKFRPKGKP

SEQ ID NO: 54 - protein, 17 aa, Melanoma Associated Endogenous Retrovirus
DFSYQRSLKFRPKGKPC

SEQ ID NO: 55 - protein, 17 aa, Melanoma Associated Endogenous Retrovirus
FSYQRSLKFRPKGKPCP

SEQ ID NO: 56 - protein, 17 aa, Melanoma Associated Endogenous Retrovirus
SYQRSLKFRPKGKPCPK

SEQ ID NO: 57 - protein, 17 aa, Melanoma Associated Endogenous Retrovirus
QRSLKFRPKGKPCPKEI

SEQ ID NO: 58 - protein, 17 aa, Melanoma Associated Endogenous Retrovirus
RSLKFRPKGKPCPKEIP

SEQ ID NO: 59 - protein, 17 aa, Melanoma Associated Endogenous Retrovirus
SLKFRPKGKPCPKEIPK

SEQ ID NO: 60 - protein, 17 aa, Melanoma Associated Endogenous Retrovirus
LKFRPKGKPCPKEIPKE

SEQ ID NO: 61 - protein, 17 aa, Melanoma Associated Endogenous Retrovirus
KFRPKGKPCPKEIPKES

SEQ ID NO: 62 - protein, 17 aa, Melanoma Associated Endogenous Retrovirus
RPKGKPCPKEIPKESKN

SEQ ID NO: 63 - protein, 17 aa, Melanoma Associated Endogenous Retrovirus
PKGKPCPKEIPKESKNT

SEQ ID NO: 64 - protein, 17 aa, Melanoma Associated Endogenous Retrovirus
KGKPCPKEIPKESKNTE

SEQ ID NO: 65 - protein, 17 aa, Melanoma Associated Endogenous Retrovirus
KPCPKEIPKESKNTEVL

SEQ ID NO: 66 - protein, 17 aa, Melanoma Associated Endogenous Retrovirus
PCPKEIPKESKNTEVLV

SEQ ID NO: 67 - protein, 17 aa, Melanoma Associated Endogenous Retrovirus
CPKEIPKESKNTEVLVW

SEQ ID NO: 68 - protein, 17 aa, Melanoma Associated Endogenous Retrovirus
PKEIPKESKNTEVLVWE

SEQ ID NO: 69 - protein, 20 aa, Melanoma Associated Endogenous Retrovirus
SYQRSLKFRPKGKPCPKEIP

SEQ ID NO: 70 - protein, 6 aa, Melanoma Associated Endogenous Retrovirus
EMQRKA

SEQ ID NO: 71 - protein, 14 aa, Melanoma Associated Endogenous Retrovirus
MQRKAPPRRRRHRN

SEQ ID NO: 72 - protein, 7 aa, Melanoma Associated Endogenous Retrovirus
RKAPPRR

SEQ ID NO: 73 - protein, 11 aa, Melanoma Associated Endogenous Retrovirus
KAPPRRRRHRN

SEQ ID NO: 74 - protein, 9 aa, Melanoma Associated Endogenous Retrovirus
RRRRHRNRA

SEQ ID NO: 75 - protein, 6 aa, Melanoma Associated Endogenous Retrovirus
YQRSLK

SEQ ID NO: 76 - protein, 12 aa, Melanoma Associated Endogenous Retrovirus
QRSLKFRPKGKP

SEQ ID NO: 77 - protein, 10 aa, Melanoma Associated Endogenous Retrovirus
RSLKFRPKGK

SEQ ID NO: 78 - protein, 12 aa, Melanoma Associated Endogenous Retrovirus
SLKFRPKGKPCP

SEQ ID NO: 79 - protein, 9 aa, Melanoma Associated Endogenous Retrovirus
FRPKGKPCP

SEQ ID NO: 80 - protein, 7 aa, Melanoma Associated Endogenous Retrovirus
KGKPCPK

SEQ ID NO: 81 - protein, 7 aa, Melanoma Associated Endogenous Retrovirus
GKPCPKE

SEQ ID NO: 82 - protein, 7 aa, Melanoma Associated Endogenous Retrovirus
PCPKEIP

SEQ ID NO: 83 - protein, 7 aa, Melanoma Associated Endogenous Retrovirus
EIPKESK

SEQ ID NO: 84 - protein, 14 aa, Melanoma Associated Endogenous Retrovirus
KGKPCPKEIPKESK

SEQ ID NO: 85 - protein, 7 aa, Melanoma Associated Endogenous Retrovirus
FSYQRSL

SEQ ID NO: 86 - protein, 11 aa, Melanoma Associated Endogenous Retrovirus
SYQRSLKFRPK

SEQ ID NO: 87 - protein, 9 aa, Melanoma Associated Endogenous Retrovirus
YQRSLKFRP

SEQ ID NO: 88 - protein, 7 aa, Melanoma Associated Endogenous Retrovirus
RSLKFRP

SEQ ID NO: 89 - protein, 9 aa, Melanoma Associated Endogenous Retrovirus
KGKPCPKEI

SEQ ID NO: 90 - protein, 13 aa, Melanoma Associated Endogenous Retrovirus
FRPKGKPCPKEIP

SEQ ID NO: 91 - protein, 10 aa, Melanoma Associated Endogenous Retrovirus
GKPCPKEIPK

(19) United States
(12) Patent Application Publication
(10) Pub. No.: US 2010/0285509 A1
(43) Pub. Date: Nov. 11, 2010

(54) Melanoma-Associated Endogenous Retrovirus (MERV) Derived Peptide Sequences And Their Therapeutic/Diagnostic Use

(76) Inventors: Bernd Mayer, Vienna (AT); Johannes Humer, Vienna (AT); Andrea Waltenberger, Horn (AT); Thomas Muster, Vienna (AT)

Correspondence Address: FULBRIGHT & JAWORSKI L.L.P., 600 CONGRESS AVE., SUITE 2400, AUSTIN, TX, 78701, US

(21) Appl. No.: 12/768,076

(22) Filed: Apr. 27, 2010

Related U.S. Application Data
(62) Division of application No. 11/914,098, filed on Jun. 13, 2008.

(30) Foreign Application Priority Data
May 11, 2005 (AT) A 807/2005

(51) Int. Cl. G01N 33/53; C07K 7/06; C07K 7/08

(52) U.S. Cl. 435/7.92; 530/329; 530/328; 530/326; 530/327; 436/501

(57) ABSTRACT

The present invention provides antigenic polypeptides derived from the melanoma-associated endogenous retrovirus (MERV). These antigens are useful compounds for the detection of cancerous cells and melanoma-diagnosis as well as melanoma-prognosis. Furthermore these antigenic polypeptides of the present invention form the basis for anti-cancer vaccines.

This application is a divisional of U.S. application Ser. No. 11/914,098 filed on Jun. 13, 2008, which is a national phase application under 35 U.S.C. §371 of International Application No. PCT/AT2006/000197 May 11, 2006, which claims priority to Austrian Patent Application No. A 807/2005 filed May 11, 2005. The entire text of each of the above-referenced disclosures is specifically incorporated herein by reference.

The present invention relates to cancer-related human endogenous retroviruses and antigenic fragments thereof. Applications for melanoma diagnosis and prognosis, as well as for vaccines and immunotherapies are presented.

Human endogenous retroviral sequences (HERVs) are possible pathogens in carcinogenesis. The human genome contains about 5% of endogenous retroviral sequences (Venter et al.). The human endogenous retrovirus type K (HERV-K) comprises 30-50 full-length members per haploid genome and shows intact open reading frames for the gag, pol and env genes. Although most of the HERV proviruses contain deletions, stop codons or frame shifts, HERV-K is one of the best described human endogenous retroviruses with open reading frames for the structural and enzymatic proteins gag, prt, pol and env (Lower et al, Mayer et al). The HERV-K (HML-2) group has also been shown to form viral particles (Bronson, Lower, Turner).

Endogenous retroviruses are frequently reported to be associated with tumour formation. A 80 kDa protein related to gag polyprotein has been identified in teratocarcinoma cell lines and in human germ cell tumours. The high expression observed in these cells is associated with the presence of antibodies directed against its retroviral products in patients with germ cell tumours (Sauter et al). Recently HERV-K gag/env antibodies have been characterised as indicators for therapy effects in patients with germ cell tumours (Kleiman et al.). Boller et al. demonstrated that HTDV particles are expressed in vivo and that the immune reaction against HTDV/HERV-K is specific for defined viral proteins. High antibody titers were found in about 60% of male patients with germ cell tumours. Antibody reactivity declined after tumour removal. Goedert et al. described that HERV-K10 anti-bodies are detected frequently with testicular cancer and seem to resolve rapidly with effective therapy of the malignancy. Anti-body reactivity also occurs in approximately 5% of controls, perhaps because of nonspecific or cross-reactive epitopes. Using realtime RT-PCR overexpression of HERV-K10-like gag genes in the blood cells of leukemia patients was shown (Depil et al.). In addition, autoantibodies to HERV-K in autoimmune diseases have been described (Herve et al.) and IgG-antibodies against murine leukemia virus were detected in psoriasis (Moles et al.).

It was reported recently that retroviral proteins and particles are specifically expressed in human melanomas and metastases but not in melanocytes (Muster et al.). Because of a sequence homology of 98% to corresponding regions of endogenous retrovirus HERV-K 108, the name MERV (melanoma-associated endogenous retrovirus) was used. The data suggest that expression of the viral sequences is activated during transformation of melanocytes to melanoma cells.

Melanoma is a cancer of the skin, up to 30% of the patients will develop systemic metastasis and the majority will die (Kirkwood et al.). Classic modalities of treating melanoma include surgery, radiation and chemotherapy. In the past decade immunotherapy and gene therapy have emerged as new and promising methods for treating melanoma. Therefore optimised antigens with specific B- and T-cell epitopes are sought after.

Different antigenic peptides are disclosed in WO 03/018610, which are used for the treatment of melanoma patients. These peptides are derived from the melanocyte differentiation antigen, gp100, which is expressed in more than 75% of human melanomas.

JP 2002/223765 A provides a malignant melanoma antigen obtained from a malignant melanoma cell line by cDNA techniques.

Further melanoma-associated antigen-like peptides, expressed in approximately 40% of melanomas and located in the Xp arm of the X chromosome, are presented in WO 02/059314.

WO 01/14884 discloses an epitope of a high molecular weight melanoma associated antigen (HMW-MAA) displayed on the surface of human cells.

WO 00/24778 describes epitopes of the melanoma antigen tyrosinase-related protein 2.

Further antigens or melanoma-derived epitopes are disclosed in WO 98/55133, WO 97/39774, U.S. Pat. No. 6,500,919, WO 95/04542, WO 92/21767 or WO 89/11296.

WO 02/046477 discloses HERV sequences including sequences for the gag, env and pol amino acid sequences from HERV.

WO 03/029460 (included by reference) describes a MERV (NCBI accession number: AX743231) and provides sequences for the gag, env and pol genes, as well as antigenic fragments thereof.

The objective of the present invention is to identify MERV-specific antigens and epitopes for the detection of melanoma and metastases thereof. Appropriate antigens for the detection of melanomas can be identified by assays using sera of melanoma patients. The specific expression of retroviral proteins in melanomas and the presence of antibodies to these proteins in melanoma patients indicate that the corresponding antigens represent targets for both immunotherapy and diagnosis.

Therefore, the present invention provides an antigen being a fragment of an amino acid sequence of the env- or gag-protein of the melanoma-associated endogenous retrovirus (MERV), comprising any one of the amino acid sequences EMQRKAPPRRRRHRNRA (SEQ ID NO 1), YQRSLKFRPKGKPCPKE (SEQ ID NO 7), FRPKGKPCPKEIPKESK (SEQ ID NO 8), FSYQRSLKFRPKGKPCP (SEQ ID NO 55), SYQRSLKFRPKGKPCPK (SEQ ID NO 56), QRSLKFRPKGKPCPKEI (SEQ ID NO 57), RSLKFRPKGKPCPKEIP (SEQ ID NO 58), SLKFRPKGKPCPKEIPK (SEQ ID NO 59) or SYQRSLKFRPKGKPCPKEIP (SEQ ID NO 69). The antigenic properties of peptides comprising the sequences of SEQ ID NOs 1, 7, 8, 13, 21, 55-59 or 69 was tested and verified using the methods disclosed in the examples. The results are given in example 11 below. Thereby, the antigenic activity of the peptides with the amino acid sequences of SEQ ID NOs 1, 7, 8, 13, 21, 55-59 and 69 against antibody containing sera of melanoma patients has been proven and thus these antigens or antigenic peptides are provided by the present invention.

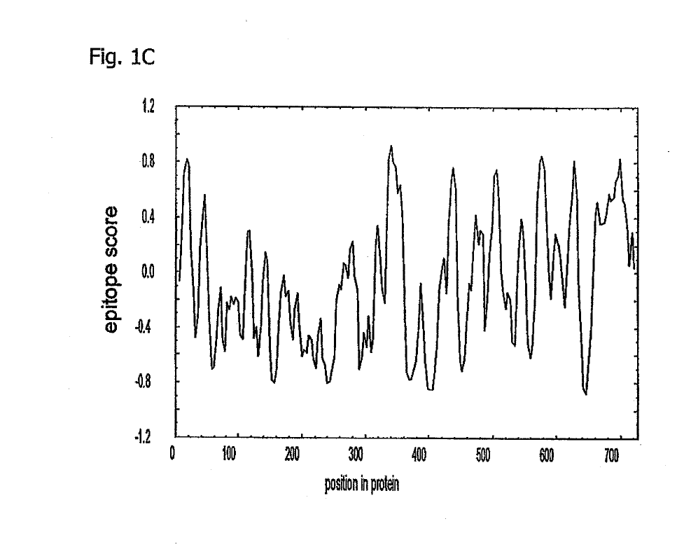

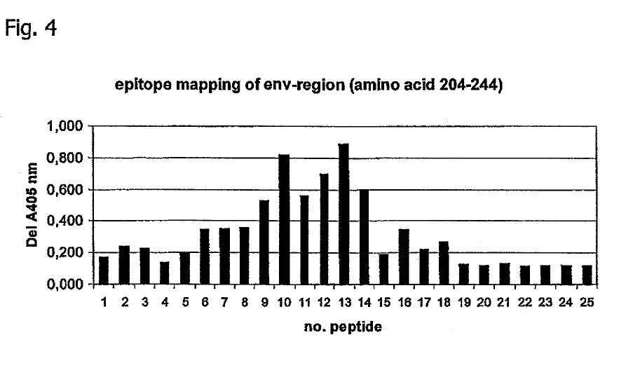

In addition to the above mentioned antigens, which showed excellent antigenic properties, the present invention also includes an antigen being a fragment of an amino acid sequence of the melanoma-associated endogenous retrovirus MERV, comprising any one of the amino acid sequences of RMKLPSTKKAEPPTWAQ (SEQ ID NO 2), TKKAEPPTWAQLKKLTQ (SEQ ID NO 3), MPAGAAAANYTYWAYVP (SEQ ID NO 4), PIDDRCPAKPEEEGMMI (SEQ ID NO 5), YPPICLGRAPGCLMPAV (SEQ ID NO 6), GKPCPKEIPKESKNTEV (SEQ ID NO 9), GTIIDWAPRGQFYHNCS (SEQ ID NO 10), RGQFYHNCSGQTQSCPS (SEQ ID NO 11), DLTESLDKHKHKKLQSF (SEQ ID NO 12), PWGWGEKGISTPRPKIV (SEQ ID NO 13), PKIVSPVSGPEHPELWR (SEQ ID NO 14), CPWFPEQGTLDLKDWKR (SEQ ID NO 15), IGKELKQAGRKGNIIPL (SEQ ID NO 16), DCNENTRKKSQKETEGL (SEQ ID NO 17), TLKLEGKGPELVGPSES (SEQ ID NO 18), GPSESKPRGTSPLPAGQ (SEQ ID NO 19), QPQTQVKENKTQPPVAY (SEQ ID NO 20), PAELQYRPPPESQYGYP (SEQ ID NO 21), MPPAPQGRAPYPQPPTR (SEQ ID NO 22), EIIDKSRKEGDTEAWQF (SEQ ID NO 23), MPPGEGAQEGEPPTVEA (SEQ ID NO 24), MKEGVKQYGPNSPYMRT (SEQ ID NO 25), VQEQVQRNRAANPPVNI (SEQ ID NO 26), LRAWEKIQDPGSTCPSF (SEQ ID NO 27), TVRQSSKEPYPDFVARL (SEQ ID NO 28), QSAIKPLKGKVPAGSDV (SEQ ID NO 29), TGREPPDLCPRCKKGKH (SEQ ID NO 30), LSGNEQRGQPQAPQQTG (SEQ ID NO 31), QPFVPQGFQGQQPPLSQ (SEQ ID NO 32), QLPQYNNCPPPQAAVQQ (SEQ ID NO 33), AINNKEPATRFQWKVLP (SEQ ID NO 34), ENRKIKPQKIEIRKDTL (SEQ ID NO 35), ILPKITRREPLENALTV (SEQ ID NO 36), FTDGSSNGKAAYTGPKE (SEQ ID NO 37), PKERVIKTPYQSAQRAE (SEQ ID NO 38), LPGPLTKANEEADLLVS (SEQ ID NO 39), LKNKFDVTWKQAKDIVQ (SEQ ID NO 40), PTQEAGVNPRGLCPNAL (SEQ ID NO 41), IWATCQTGESTSHVKKH (SEQ ID NO 42), VPEKIKTDNGPGYCSKA (SEQ ID NO 43), LVKQKEGGDSKECTTPQ (SEQ ID NO 44), AEQHLTGKKNSPHEGKL (SEQ ID NO 45), IWWKDNKNKTWEIGKVI (SEQ ID NO 46), PRVNYLQDFSYQRSLKF (SEQ ID NO 47), RVNYLQDFSYQRSLKFR (SEQ ID NO 48), VNYLQDFSYQRSLKFRP (SEQ ID NO 49), NYLQDFSYQRSLKFRPK (SEQ ID NO 50), YLQDFSYQRSLKFRPKG (SEQ ID NO 51), QDFSYQRSLKFRPKGKP (SEQ ID NO 53), DFSYQRSLKFRPKGKPC (SEQ ID NO 54), LKFRPKGKPCPKEIPKE (SEQ ID NO 60), KFRPKGKPCPKEIPKES (SEQ ID NO 61), RPKGKPCPKEIPKESKN (SEQ ID NO 62), PKGKPCPKEIPKESKNT (SEQ ID NO 63), KGKPCPKEIPKESKNTE (SEQ ID NO 64), KPCPKEIPKESKNTEVL (SEQ ID NO 65), PCPKEIPKESKNTEVLV (SEQ ID NO 66), CPKEIPKESKNTEVLVW (SEQ ID NO 67), PKEIPKESKNTEVLVWE (SEQ ID NO 68). These antigens also showed an antigenicity clearly above the threshold of the non-antigenic controls: (see FIGS. 2 and 4).

It is known that the minimal size of a continuous epitope is 6 amino acid residues (King et al., 1994). Although epitopes can be formed by different, not directly connected amino acids in larger peptides, in smaller peptides the epitope, i.e. the part of the peptide that interacts with an antibody, is a small sequence of continuous amino acids. Therefore, the antigens of the present invention also include any fragment of an amino acid sequence of the env- or gag-protein of the melanoma-associated endogenous virus (MERV), comprising a fragment of at least 6 continuous amino acids of any one of the amino acid sequences of SEQ ID NOs 1, 7, 8, 55-59 or 69. Preferred fragments have a length of at least 8 amino acids. The preferred fragments may have a length between 8 and 15, especially between 8 to 12 amino acids. Such small peptides can be used, for example, to map the antigen-binding specificity of antibodies in a patient with melanoma for better classification of the cancer.

Preferred fragments are EMQRKA, MQRKAPPRRRRHRN, RKAPPRR, KAPPRRRRHRN, RRRRHRNRA (contained in SEQ ID NO. 1), YQRSLK, QRSLKFRPKGKP, RSLKFRPKGK, SLKFRPKGKPCP, FRPKGKPCP, KGKPCPK, GKPCPKE (contained in SEQ ID NO. 7), GKPCPKE, PCPKEIP, EIPKESK, KGKPCPKEIPKESK (contained in SEQ ID NO. 8), FSYQRSL, SYQRSLKFRPK, YQRSLKFRP, RSLKFRP (contained in SEQ ID NO. 55), KGKPCPKEI, FRPKGKPCPKEIP, GKPCPKEIPK (contained in SEQ ID NO. 59).

In specific embodiments the MERV sequences (SEQ ID NOs 1-69), or given fragments, are the only MERV-sequences (and HERV-sequences) of the antigens. This allows the production of specific antibodies without or reduced cross-reactivity. In other embodiments only 2, 3, 4, 5, 6, 7 or 8 given MERV sequences are comprised in the antigen.

Further antigens or antigenic compounds are mimotopes of the above mentioned antigens. The term “mimotopes” refers to peptides which mimic the polypeptides as defined above immunologically. Since sequence variability may occur in MERV (since it is related to cancerous mutations), it may be desirable to vary one or more amino acids so as to better mimic the epitopes of different MERV mutants, even with a different immunhistology. It should be understood that such mimotopes need not be identical to any particular MERV sequence as long as the subject compounds are capable of providing for immunological stimulation after which the T and B cells are MERV reactive (specifically, the naturally occurring homologues of MERV-antigen sequences corresponding to the SEQ ID Nos referred to above are preferred). The polypeptides as described above, may therefore be subject to insertions, deletions and conservative as well as non-conservative amino acid substitutions where such changes might provide for certain advantages in their use. Also non-natural amino acid residues (i.e. amino acid residues other than the 20 standard amino acids, such as D-amino acids, ornithine, 3- or 4-OH-proline, norvaline, norleucine, etc.) or chemically altered amino acid residues may be applied. The peptides will preferably be as short as possible while still maintaining all of their sensitivity of the larger sequence. In certain cases, it may be desirable to join two or more peptides into a single structure. The formation of such a composite may involve covalent or non-covalent linkages. The mimotope may be identified with a (monoclonal) antibody and (commercially available) peptide libraries (e.g. according to Reineke et al. 2002: “Identification of distinct antibody epitopes and mimotopes from a peptide array of 5520 randomly generated sequences” J Immunol Methods 267:37). Thus the present invention also relates to an antigen comprising a mimotope of any antigen as defined above.

Current assay techniques for the detection of antigens or antibodies employ pre-prepared (competitive) antigens. Such antigens are preferably provided immobilised onto a solid support. A common method for immobilisation is to provide antigens with a biotin-linker which can be easily bound to surface-structures (e.g. avidin) of a surface (e.g. a microtiter well, or biochip surface for microarrays). Therefore, the present invention also includes antigens, as defined above, comprising covalently bound biotin. For better epitope recognition by the antibody a linker molecule between the surface and the antigen can be used to increase the flexibility and possible modes of orientation of the antigen.

Small epitopes can be recognised by antibodies but are by themselves not antibody inducing, i.e. they do not induce the formation of specific antibodies. However, the antigen according to the present invention may also be provided or tested with respect to its T cell reactivity. Moreover, an antigen of the present invention can be provided as a protein aggregate or conjugate comprising a non-antigenic protein and an antigen of the present invention. Such an aggregate can be used to produce anti-sera or for an immunotherapy. Non-antigenic compounds are known in the state of the art and include blood compounds such as albumin.

Large immunogenic compounds can also be produced as fusion proteins comprising a non-antigenic protein and an antigen according to the present invention. The advantage in fusion proteins lies in the covalent association of the antigen and the non-antigenic protein which provides additional stability. Furthermore, such a fusion protein can be produced recombinantly by standard microbiological techniques.

A further aspect of the present invention is an antiserum comprising antibodies against an antigen or protein aggregate or fusion protein as noted above. Antisera are commonly produced by repeated antigen injection (e.g. 2 or 3 times) in an animal such as mice, rat, rabbit, guinea pig, chicken, goat, sheep, horse or cow and subsequent gathering of sera from the animal (e.g. by bleeding or gathering of eggs). An antiserum produced in this way is a polyclonal antiserum, i.e. several types of antibodies recognising the same antigen may be present in the serum. Such antisera can optionally be enriched in antigen-specific antibodies by immunoadsorption and desorption on a column or beads comprising the subject antigen, i.e. an antigen as defined above. Such antisera can be used for the detection of MERV antigens in a sample by standard assay methods. Antisera may comprise preservatives such as timerosal or sodium azide.

Furthermore, an isolated antibody directed against an anti-gen or protein aggregate or fusion protein as defined above is provided, which can be used for various assay and detection techniques related to MERV analysis, wherein MERV antibodies in patient represent a diagnostic indicator for melanoma. Such an antibody can be obtained from a polyclonal antiserum by an affinity assay or alternatively monoclonal antibodies can be produced using the hybridoma method (Barnstable et al.).

Furthermore, the present invention provides a method for the detection of anti-MERV-antibodies in a sample using an antigen according to the present invention comprising the steps of
(a) contacting said sample with said antigens, which leads to an antibody-antigen reaction between said antibody from the sample and said antigen, and
(b) detecting and optionally quantifying said anti-MERV anti-body by said binding to said antigen.
   Such detection methods are common knowledge in the state of the art of immuno assays.

Preferably the above method for the detection of anti-MERV-antibodies is used simultaneously for the quantification of said anti-MERV-antibodies, wherein the anti-MERV antibody is quantified by either determining the amount of antibody-bound antigen, or the amount of antigen-bound antibody, or the amount of antibody-free antigen, or the amount of antigen-free antibody.

In a preferred method for the detection of anti-MERV-antibodies as described above the antigen is immobilised on a surface.

A further aspect of the method for the detection of anti-MERV-antibodies estimates the amount of antibody-free antigen by at least one additional secondary antibody, which creates a detectable marker signal. Secondary antibodies are used to detect primary antibodies by binding to the constant part or Fc part of the primary antibody. This is a common set-up for immuno assays, especially competitive immuno assays.

A preferred method according to the invention is an enzyme-linked immunosorbent assay (ELISA), wherein the detected signal is amplified by an enzymatic reaction of an enzyme covalently linked to a (secondary) antibody.

Since HERV or MERV proteins are not expressed under normal circumstances, the presence of MERV antigens and anti-MERV-antibodies in a patient are indicators for melanoma. Therefore the present invention includes a method for the diagnosis of melanoma, wherein an antibody is detected as described above, wherein the presence of such an antibody is an indicator of melanoma. Therefore the present invention relates to a method for melanoma diagnosis using an antigen according to the invention comprising the steps of
(a) contacting a sample with said antigens, which leads to an antibody-antigen reaction between antibodies from the sample and said antigen, and
(b) detecting and optionally quantifying said anti-MERV anti-body by said binding to said antigen, wherein the presence of antigens indicates melanoma.

A further aspect of the invention is a method for the detection of a MERV protein or MERV protein fragment in a sample using an antibody or antibody fragment, which is directed against an antigen as defined above, comprising the steps of
(a) contacting said sample with said antibody, which leads to an antibody-antigen reaction between said antibody and said MERV protein or MERV protein fragment, and
(b) either determining the amount of antibody-bound MERV protein or MERV protein fragment, or the amount of MERV protein- or MERV protein fragment-bound antibody, or the amount of antibody-free MERV protein or MERV protein fragment, or the amount of MERV protein- or MERV protein fragment-free antibody.
   The presence of such a protein or protein fragment in a sample obtained from a patient is an indicator of melanoma.

Preferably, the method as noted above utilises an antigen as defined above as competitive antigen.

Even further preferred is the immobilisation of the antigen onto a surface of said competitive antigen for easier phase separation during an immunoassay.

As noted above the detection of MERV-antigens or MERV-directed antibodies can be used for the diagnosis of melanoma or melanoma cells. Therefore the current invention also relates to a method for diagnosing cancerous cells comprising the steps of
(a) providing a sample of said cells to be tested or supernatant thereof,
(b) analysing whether or not an antigen as defined above is present in said sample whereby
(c) the presence of said antigen in said sample diagnoses cancerous cells.

Although MERV associated antigens are present in a patient with melanoma, such antigens are likely to be expressed even before the cancer becomes malignant. The expression of MERV proteins may be the cause of melanoma since retroviral actions, such as reverse transcription and insertions of the viral genome into different locations of the host cell promote the cancer. Therefore the presence of MERV antigens can also indicate precancerous cells, whereby the method for the detection of MERV-associated antigens or anti-MERV-antibodies can be used for the diagnosis or prognosis of cancer, preferably melanoma.

The antigens of the present invention can also be used to stimulate the immune response in a patient prior to cancer or after melanoma emergence. The invention provides a pharmaceutical composition comprising an antigen or an antigenic protein aggregate or an antigenic fusion protein as noted above.

Such a pharmaceutical composition can further comprise a pharmaceutical carrier and/or an adjuvant. Such pharmaceutical carriers are for example stabilising salts, emulgators, solubilisers or osmo-regulators, suspending agents, thickening agents, redox components maintaining a physiological redox potential. Preferred adjuvants include aluminium salts, microemulsions, lipid particles, oligonucleotides such as disclosed in Singh et al. and are used to increase the immune response.

A further aspect of the present invention is a pharmaceutical composition or preparation as vaccine comprising an antigen or an antigenic protein aggregate or an antigenic fusion protein as noted above. A vaccine can be used for an injection as treatment of melanoma or prevention of melanoma.

An even further aspect of the present invention is a kit for carrying out a method for the detection of MERV antigens or anti-MERV-antibodies in a sample comprising an antigen as defined above, a first antibody directed against said antigen, a marker-linked secondary antibody directed against the Fc region of said first antibody, buffer substances, positive control standards, which are compositions containing a protein or protein fragment of MERV, and negative control standards, which are compositions containing a protein or protein fragment not encoded by the MERV genome.

A further aspect of such a kit provides the antigen of the present invention immobilised onto a solid support, such as microtiter wells or biochips for microarrays.

The present invention is described in more detail with the help of the following examples and figures to which it should, however, not be limited.

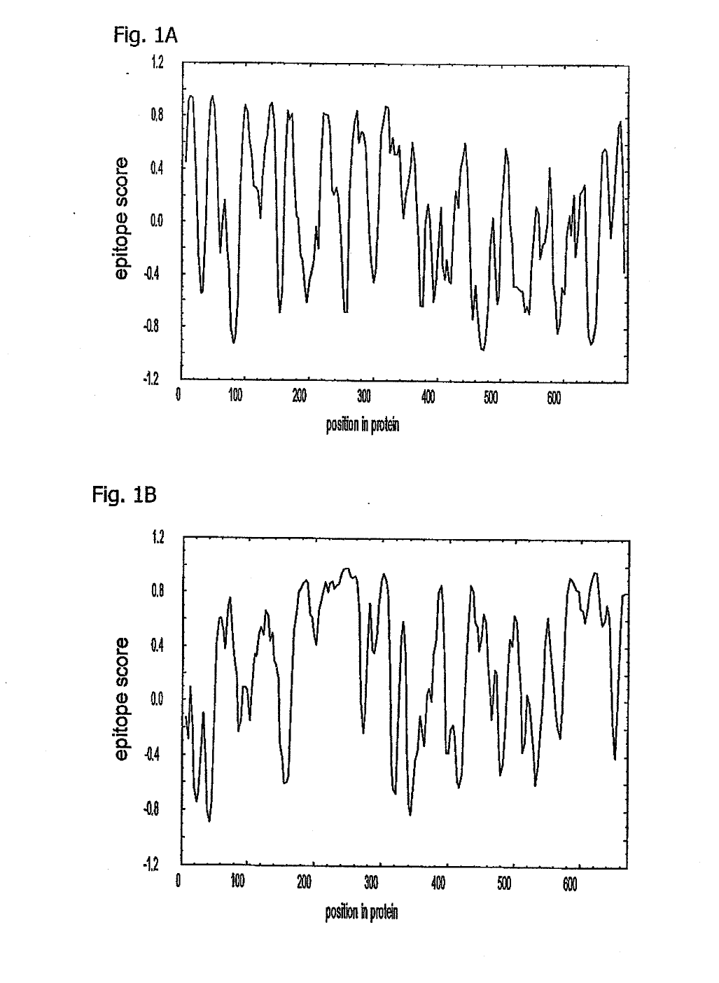

FIG. 1: Antigenicity profiles of env (A), gag (B)), and pol (C). The x-axis represents the position within each protein (starting at the N-terminus with residue one). The y-axis displays the E-Score predictions, i.e. the epitope scores, providing distinct values for each amino acid along the sequence, normalised to the interval [−1,1].

FIG. 2: Selected candidate peptides by Epitope prediction were tested with a melanoma sera pool and a reference sera pool respectively. The mouse derived control peptides K1 (Biotin-SGSG-KPLAQ-NH2) and K2 (Biotin-SGSG-GLAQ-NH2) were used as negative and positive control peptides. ELISA readout of patient sera pool (black bars) given as absorbance determined at 405 nm. All given A405 nm values refer to the measured A405 nm value of each sample minus the blank.

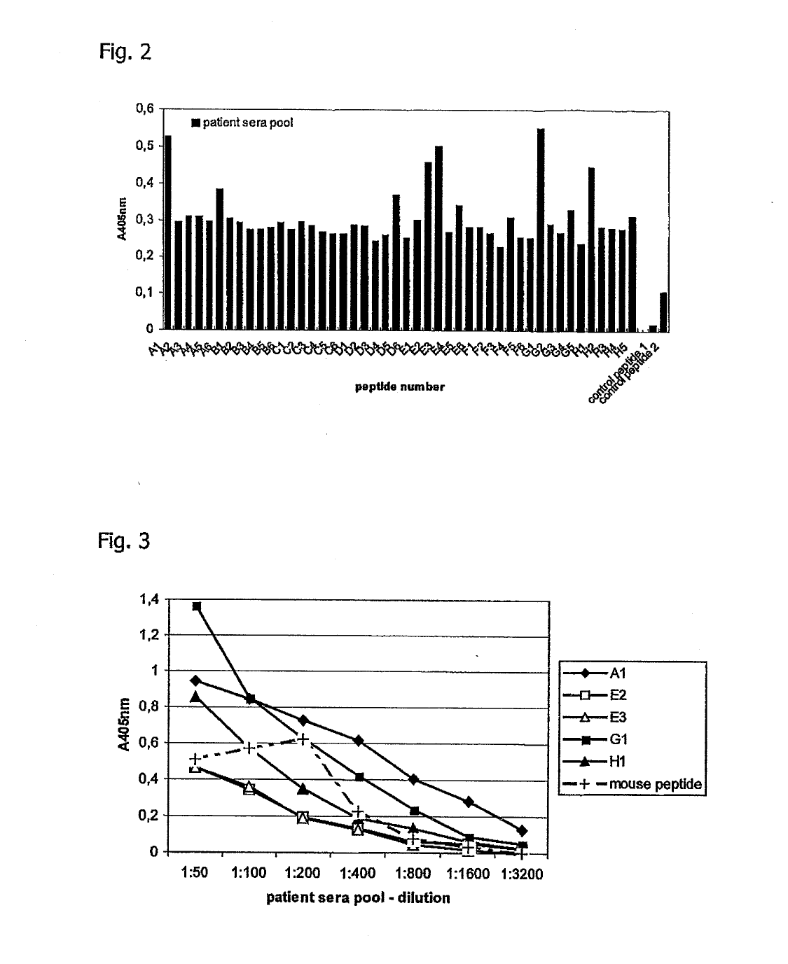

FIG. 3: Response of melanoma-sera pool to 5 preselected antigens. The plates were coated with the antigens (A1, E2, E3, G1, H1), and serial dilutions of melanoma-sera pool were added to wells. Dilutions were done using the reference-sera pool and a mouse peptide was used as negative control peptide. One experiment of two performed is shown. Mean values from duplicate trials are shown.

FIG. 4: Epitope mapping of 25 overlapping env-peptides tested with a patient sera pool and a reference sera pool as described above. The first bar represents amino acid 204-220, the second bar represents amino acid 205-221, etc. Del A405 nm refers to the measured A405 nm values of the melanoma sera pool minus the A405 nm values of the reference sera pool of each peptide. One experiment of two performed is shown. Mean values from duplicate trials are shown.

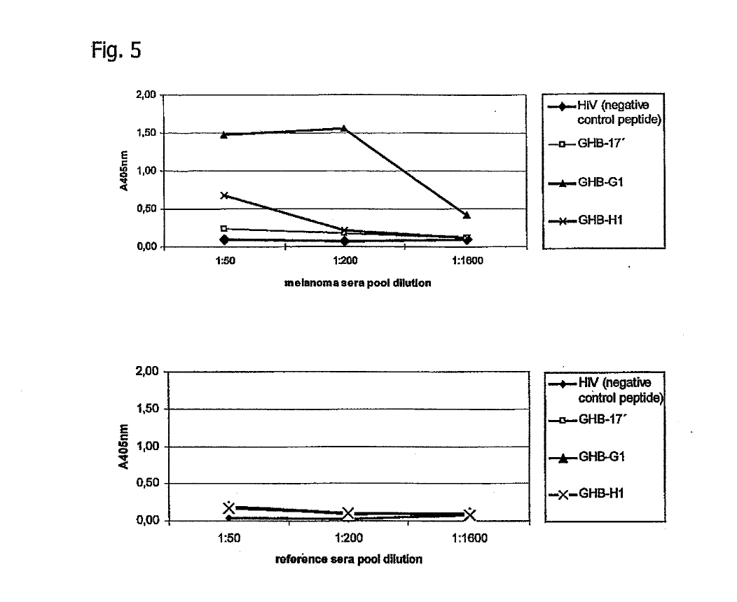

FIG. 5: Reactivity of serum antibodies with 2 MERV specfic partial overlapping peptides (GHB-G1 and GHB-H1) and 1 autoimmune-related peptide (GHB-17′) tested with 3 different melanoma-sera and reference sera pool dilutions respectively. An HIV peptide was used as negative control peptide. All given A405 nm values refer to the measured A405 nm value of each sample minus the blank.

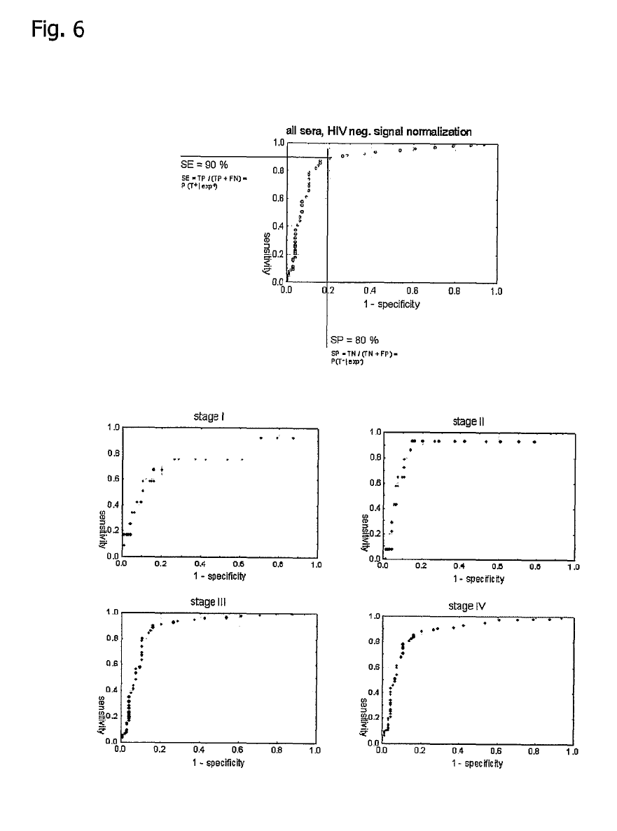

FIG. 6: Preliminary data analysis was performed to reveal general sensitivity and specificity. The receiver-operating characteristic (ROC) curve was used to evaluate the diagnostic value of melanoma patient sera and to define the optimal cut-off point for the readout value that corresponds to the highest accuracy of discrimination between melanoma and non-melanoma patients. Mean values of triplicate measurements were used. To compute ROC curves each plate was normalised with respect to the mean signal of the per plate HIV control wells. ROC curves were generated by computing FP, FN, TP, TN at diverse signal difference cut-off values with respect to background. In total 100 cut-offs were chosen (equidistant intervals given in-between the minimum and maximum signal readout).

Sensitivity was computed as: SE=TP/(TP+FN)=P(T+|exp+)

Sensitivity therefore defines the probability of a positive test when a positive experiment is given (i.e. melanoma sera). The number of false negatives decreases the text sensitivity.

Specificity was computed as: SP=TN/(TN+FP)=P(T−|exp−)

Specificity therefore defines the probability of a negative test when a negative experiment is given (i.e. reference sera). The number of false positives decreases the test specificity.

The following number of sera was used for the analysis (sera with unclear staging were not further considered):

Stage I: 12

Stage II: 14

Stage III: 204

Stage IV: 136

Reference: 95

Analysing the ROC curve for all sera reveals a readout cut-off where SE reaches 90% and SP reaches 80%. SE and SP are comparable for stage II, III, and IV. The respective values are significantly lower for stage I sera. This may be based on the small number of sera given, or on the biology, e.g. insufficient Breslow hindering a presentation of epitopes to the immune system.

### EXAMPLES

The following examples specify a method for the detection of short peptides corresponding to B-cell epitopes of MERV, predicted by the program E-Score. Predicted peptides were analyzed for their reactivity to pools of sera derived from melanoma patients. Immunodominant peptides located in the env protein of MERV were identified.

### Example 1

### Epitope Prediction

Short amino acid sequences of MERV (NCBI accession number: AX743231) were identified during evaluation runs using the E-score programme for sequence analysis and epitope prediction.

### Example 2

### Epitope Selection

Gag, pol and env proteins were analysed for the presence of potential B-cell epitopes. Epitope selection was based on the E-Score predictions. FIG. 1 shows the computed antigenicity profiles for env (699 aa), gag (670 aa), and pol (726 aa), Peptides (17-mers) corresponding to peaks showing E-Score values equal or above 0.8 were selected for the subsequent prescreening. This cut-off was used as E-Score validation experiments revealed Positive Predictive Values of about 80% at that particular prediction cut-off. In case prediction revealed broad peak areas, overlapping peptides were selected to cover the whole area of interest. In itotal 14 env-derived peptides, 19 gag-derived peptides, and 13 pol sequences were selected, synthesised and tested.

### Example 3

### Immune Sera

Serum specimens were collected from melanoma patients (diagnosis confirmed by histopathology) at the Department of Dermatology, Medical University of Vienna, Austria. Staging of patients and according classification of sera followed the 2001 US Joint Committee on Cancer guidelines (Balch 2001). Usage of patient sera was approved by the ethical committee of the Medical University of Vienna, confidentially of the study subjects has been protected by respective sample coding. Sera from healthy donors served as negative controls. All sera were stored at −20° C. immediately after blood withdrawal. Melanoma patient derived sera pools and respective reference sera pools from healthy subjects were used for epitope screening and further peptide testing. Sample size was 10 sera from different melanoma patients exhibiting stage III and IV at the time point of blood withdrawal (melanoma-sera pool), and 10 sera from healthy subjects respectively (reference sera pool).

### Example 4

### Peptide Synthesis

Peptides selected based on the E-Score prediction scores were synthesised (PERBIO Science, The Netherlands) at 80% purity. 3-5 mg of synthesised biotinylated peptide were diluted in 400 μl of a 50% dimethylformamide solution. Peptides for further testing and final screening were synthesised at >90-95% purity without biotinylation (PiCHEM research and development, Graz, Austria). The purity of these peptides was assessed by HPLC and MS. Peptides were diluted with dimethylsulfoxide to a final concentration of 3 mg/ml.

### Example 5

### Epitope Screening

Streptavidin-coated 96-well microtiter plates (Mimotopes Pty Ltd., Australia) were blocked with 200 μl/well of 2% bovine albumine (Sigma-Aldrich) in PBST (PBS [0.1 M sodium phosphate, 0.15 M NaCl, pH 7.0]+0.1% v/v Tween20 (PBST)) over night at 4° C. The wells were then washed four times with PBST and incubated with 100 μl/well of 1:500 diluted biotinylated peptides for 2 hours at room temperature. 2 wells per plate were incubated with PBST in the absence of peptide (blank wells). Plates were washed 4 times with PBST. Subsequently, 100 μl of a melanoma-sera pool, diluted 1:40 in 1% bovine albumine/PBST and a reference serum pool (1:40) were added to each well and incubated for 2 h at room temperature. The plate was washed 4 times with PBST and incubated with 100 μl/well of the secondary antibody: goat anti-human IgG (h+l) antibody alkaline-phosphatase conjugated (BETHYL Laboratories, Inc., USA). Detection antibody was diluted 1:1000 in blocking solution and incubated for 1 hour. After 6 washing steps with PBST, 200 μl of a 1.0 mg/ml p-Nitrophenylphosphat substrate solution in 0.2 M Tris-buffer (Sigma-Aldrich) was added to each well. Absorbance was measured on a BDSL Immunoskan PLUS at 405 nm.

During initial screening of various peptides reactive peptides to the melanoma patient derived sera pool were selected and also validated by determining the reactivity to the reference sera pool obtained from healthy volunteers. Table 1 shows selected candidate peptides and the peptide position within the proteins env, gag, and pol. These peptides were tested to determine the experimental antigenicity.

TABLE 1
List of synthetic predicted antigenic peptides
covering the env, gag and pol region. All
peptides were experimentally tested (with
N-terminal biotin label).

| Frag- | SEQ | | | | | |
|---|---|---|---|---|---|---|
| ment | ID | | | | | pro- |
| no. | NO | peptide sequence | from | to | length | tein |
| A1 | 1 | EMQRKAPPRRRRHRNRA | 5 | 21 | 17 | Env |
| B1 | 2 | RMKLPSTKKAEPPTWAQ | 36 | 52 | 17 | Env |
| C1 | 3 | TKKAEPPTWAQLKKLTQ | 42 | 58 | 17 | Env |
| D1 | 4 | MPAGAAAANYTYWAYVP | 92 | 108 | 17 | Env |
| E1 | 5 | PIDDRCPAKPEEEGMMI | 136 | 152 | 17 | Env |
| F1 | 6 | YPPICLGRAPGCLMPAV | 160 | 176 | 17 | Env |
| G1 | 7 | YQRSLKFRPKGKPCPKE | 214 | 230 | 17 | Env |
| H1 | 8 | FRPKGKPCPKEIPKESK | 220 | 236 | 17 | Env |
| A2 | 9 | GKPCPKEIPKESKNTEV | 224 | 240 | 17 | Env |
| B2 | 10 | GTIIDWAPRGQFYHNCS | 260 | 276 | 17 | Env |
| C2 | 11 | RGQFYHNCSGQTQSCPS | 268 | 284 | 17 | Env |
| D2 | 12 | DLTESLDKHKHKKLQSF | 294 | 310 | 17 | Env |
| E2 | 13 | PWGWGEKGISTPRPKIV | 312 | 328 | 17 | Env |
| F2 | 14 | PKIVSPVSGPEHPELWR | 325 | 341 | 17 | Env |
| G2 | 15 | CPWFPEQGTLDLKDWKR | 50 | 66 | 17 | Gag |
| H2 | 16 | IGKELKQAGRKGNIIPL | 67 | 83 | 17 | Gag |
| A3 | 17 | DCNENTRKKSQKETEGL | 118 | 134 | 17 | Gag |
| B3 | 18 | TLKLEGKGPELVGPSES | 164 | 180 | 17 | Gag |
| C3 | 19 | GPSESKPRGTSPLPAGQ | 176 | 192 | 17 | Gag |
| D3 | 20 | QPQTQVKENKTQPPVAY | 198 | 214 | 17 | Gag |
| E3 | 21 | PAELQYRPPPESQYGYP | 219 | 235 | 17 | Gag |
| F3 | 22 | MPPAPQGRAPYPQPPTR | 237 | 253 | 17 | Gag |
| G3 | 23 | EIIDKSRKEGDTEAWQF | 270 | 286 | 17 | Gag |
| H3 | 24 | MPPGEGAQEGEPPTVEA | 293 | 309 | 17 | Gag |
| A4 | 25 | MKEGVKQYGPNSPYMRT | 322 | 338 | 17 | Gag |
| B4 | 26 | VQEQVQRNRAANPPVNI | 378 | 394 | 17 | Gag |
| C4 | 27 | LRAWEKIQDPGSTCPSF | 428 | 444 | 17 | Gag |
| D4 | 28 | TVRQSSKEPYPDFVARL | 446 | 462 | 17 | Gag |
| E4 | 29 | QSAIKPLKGKVPAGSDV | 493 | 509 | 17 | Gag |
| F4 | 30 | TGREPPDLCPRCKKGKH | 578 | 594 | 17 | Gag |
| G4 | 31 | LSGNEQRGQPQAPQQTG | 610 | 626 | 17 | Gag |
| H4 | 32 | QPFVPQGFQGQQPPLSQ | 631 | 647 | 17 | Gag |
| A5 | 33 | QLPQYNNCPPPQAAVQQ | 654 | 670 | 17 | Gag |
| B5 | 34 | AINNKEPATRFQWKVLP | 9 | 25 | 17 | Pol |
| C5 | 35 | ENRKIKPQKIEIRKDTL | 109 | 125 | 17 | Pol |
| D5 | 36 | ILPKITRREPLENALTV | 313 | 329 | 17 | Pol |
| E5 | 37 | FTDGSSNGKAAYTGPKE | 330 | 346 | 17 | Pol |
| F5 | 38 | PKERVIKTPYQSAQRAE | 344 | 360 | 17 | Pol |
| G5 | 39 | LPGPLTKANEEADLLVS | 433 | 449 | 17 | Pol |
| H5 | 40 | LKNKFDVTWKQAKDIVQ | 469 | 485 | 17 | Pol |
| A6 | 41 | PTQEAGVNPRGLCPNAL | 496 | 512 | 17 | Pol |
| B6 | 42 | IWATCQTGESTSHVKKH | 540 | 556 | 17 | Pot |
| C6 | 43 | VPEKIKTDNGPGYCSKA | 566 | 582 | 17 | Pol |
| D6 | 44 | LVKQKEGGDSKECTTPQ | 619 | 635 | 17 | Pol |
| E6 | 45 | AEQHLTGKKNSPHEGKL | 659 | 675 | 17 | Pol |
| F6 | 46 | IWWKDNKNKTWEIGKVI | 676 | 692 | 17 | Pol |

Based on the experimental results an epitope mapping was performed for the selected candidate area from env, G1 (aa 214-230) (see Table 2).

TABLE 2
List of synthetic peptides of the experimentally
deter-mined immunodominant part of the env
protein (amino acids 204-244).

| Peptide nr. | SEQ ID NO | Sequence | from-to |
|---|---|---|---|
| 1 | 47 | PRVNYLQDFSYQRSLKF | 204-220 |
| 2 | 48 | RVNYLQDFSYQRSLKFR | 205-221 |
| 3 | 49 | VNYLQDFSYQRSLKFRP | 206-222 |
| 4 | 50 | NYLQDFSYQRSLKFRPK | 207-223 |
| 5 | 51 | YLQDFSYQRSLKFRPKG | 208-224 |
| 6 | 52 | LQDFSYQRSLKFRPKGK | 209-225 |
| 7 | 53 | QDFSYQRSLKFRPKGKP | 210-226 |
| 8 | 54 | DFSYQRSLKFRPKGKPC | 211-227 |
| 9 | 55 | FSYQRSLKFRPKGKPCP | 212-228 |
| 10 | 56 | SYQRSLKFRPKGKPCPK | 213-229 |
| 11 = G1 | 7 | YQRSLKFRPKGKPCPKE | 214-230 |
| 12 | 57 | QRSLKFRPKGKPCPKEI | 215-231 |
| 13 | 58 | RSLKFRPKGKPCPKEIP | 216-232 |
| 14 | 59 | SLKFRPKGKPCPKEIPK | 217-233 |
| 15 | 60 | LKFRPKGKPCPKEIPKE | 218-234 |
| 16 | 61 | KFRPKGKPCPKEIPKES | 219-235 |
| 17 = H1 | 8 | FRPKGKPCPKEIPKESK | 220-236 |
| 18 | 62 | RPKGKPCPKEIPKESKN | 221-237 |
| 19 | 63 | PKGKPCPKEIPKESKNT | 222-238 |
| 20 | 64 | KGKPCPKEIPKESKNTE | 223-239 |
| 21 = A2 | 9 | GKPCPKEIPKESKNTEV | 224-240 |
| 22 | 65 | KPCPKEIPKESKNTEVL | 225-241 |
| 23 | 66 | PCPKEIPKESKNTEVLV | 226-242 |
| 24 | 67 | CPKEIPKESKNTEVLVW | 227-243 |
| 25 | 68 | PKEIPKESKNTEVLVWE | 228-244 |
| 26 (10-13) | 69 | SYQRSLKFRPKGKPCPKEIP | 213-232 |

The sequence of each peptide was represented by a series of 17-residue peptides (excluding SGSG-leader sequence) having an overlap between consecutive peptides of 16 residues.

### Example 6

### Antigen Preparation

Streptavidin-coated 96-well microtiter plates (Mimotopes Pty Ltd., Australia) were blocked with 200 μl/well of 2% bovine albumine (Sigma-Aldrich) in PBST (PBS [0.1 M sodium phosphate, 0.15 M NaCl, pH 7.0]+0.1% v/v Tween20 (PBST)) over night at 4° C. Subsequently, the wells were washed four times with PBST and incubated with 100 μl/well of biotinylated peptides A1, G1, H1, E2, E3 diluted 1:250 for 2 hours at room temperature. A mouse-specific peptide was used as negative control. The plate was washed 4 times with PBST. 2-fold serial dilutions of the melanoma sera pool containing 1% of the reference sera pool (as described above) in 1% bovine albumine/PBST were made. The plate was washed 4 times with PBST and incubated with 100 μl/well of the secondary antibody: goat anti-human IgG (h+l) antibody alkaline-phosphatase conjugated (BETHYL Laboratories, Inc., USA). Detection antibody was diluted 1:1000 in blocking solution and incubated for 1 hour. After another 6 washing steps with PBST, reaction was developed with 200 μl/well of a 1.0 mg/ml p-Nitrophenylphosphat substrate solution in 0.2 M Tris-buffer (Sigma-Aldrich). Absorbance was measured on a BDSL Immunoskan PLUS at 405 nm.

### Example 7

### Peptide Testing

NUNC Maxisorp F plates were coated with 1.0 μg peptide/well in 100 μl coating buffer (0.1 M sodium carbonate buffer, pH 9.5). 1 well per plate was coated with 100 μl of coating buffer without antigen (blank well). Plates were incubated over night at 4° C. Then plates were washed four times with PEST. Unspecific binding sites were blocked with 200 μl/well of 2% bovine albumine (Sigma-Aldrich) in PBST (PBS+0.1% v/v Tween20 (PBST)) for 1 hour at room temperature. The plates were washed 4 times with PBST. For the assay 100 μl/well of three different sera pool-dilutions (1:50, 1:200, 1:1600) diluted in 1% bovine albumine/PBST were added and incubated for 2 h at room temperature. Plates were washed 4 times with PBST and incubated with 100 μl/well of the secondary antibody: goat anti-human IgG (h+l) antibody alkaline-phosphatase conjugated, obtained from BETHYL Laboratories, Inc., USA (detection antibody was diluted 1:1000 in blocking solution) for 1 hour. After additional 6 times washing step with PBST, color was developed with 200 μl/well of a 1.0 mg/ml p-Nitrophenylphosphat substrate solution in 0.2 M Tris-buffer (Sigma-Aldrich). Absorbance was measured on a BDSL Immunoskan PLUS at 405 nm.

The peptides were tested with the same sera pools as given for the experiments above. As negative control peptide an HIV-derived peptide (GKLICTTTVPWNASWSNKSL) with 1 μg/well was used.

As shown in FIG. 2 incubating the peptides with the melanoma sera pool revealed absorption values in the range between 0.25 and 0.53. The reference sera pool revealed absorption values below 0.14. Out of these 46 tested peptides, the 5 most reactive peptides were A1, E2, E3, G1, H1 (SEQ ID NOs 1, 7, 8, 13, 21, respectively). Peptides A1, G1, H1 and E2 are derived from the env sequence, and peptide E3 is derived from the gag sequence.

FIG. 4 further demonstrates clearly that peptides 9-14 (SEQ ID NOs 55-59) have a significant higher reactivity than the other peptides indicating that this amino acid stretch represents the core epitope region. Interestingly, peptide no. 11 (G1) showed a lower absorbance in the assay than the three neighbour peptides in two independent experiments. A new synthesised unbiotinylated 20-mer peptide covering the sequence from peptides 10-13 (SYQRSLKFRPKGKPCPKEIP, SEQ ID NO 69) did not exhibit a significant improvement compared to the unbiotinylated 17-mer peptide G1 proved in an independent experiment.

### Example 8

### Screening

NUNC Polysorp F Peptide Immobiliser plates were coated with 0.125 μg peptide/well in 100 μl coating buffer ((0.1 M sodium carbonate buffer, pH 9.5). Plates were incubated over night at 4° C. Plates were then washed four times with PBST and unspecific binding sites were blocked with 200 μl/well of 2% bovine albumine (Sigma-Aldrich) in PBST (PBS+0.1% v/v Tween20 (PBST)) for 1 hour at room temperature. The plates were washed 4 times with PBST. For the assay 100 μl/well of serial-dilutions of the sera (initial dilution 1:200 in 1% bovine albumine/PBST were added and incubated for 2 h at room temperature. Plates were washed 4 times with PEST and incubated with the secondary antibody (100 μl/well). As secondary antibody an alkaline-phosphatase conjugated goat anti-human IgG (h+1) (BETHYL Laboratories, Inc., USA) diluted 1:1000 in blocking solution was used. Incubation period was 1 hour. After another 6 washing steps with PBST, the substrate was added (1.0 mg/ml p-Nitrophenylphosphat Sigma-Aldrich) in 0.2 M Tris-buffer 200 μl/well) Absorbance was measured on a BDSL Immunoskan PLUS at 405 nm.

### Example 9

### Comparison with Prior Art

Herve et al. characterise retroviral peptides in the context of autoimmune diseases. Interestingly one antigenic peptide (17′) was partially overlapping with peptide GHB-G1 and GHB-H1. All three peptides were tested with three melanoma sera pool dilutions (1:100, 1:200 and 1:1600). Peptides were coated on Nunc Maxisorp F plates and used to capture serum antibodies, which were then detected using goat anti-human IgG antibodies as shown in FIG. 5. The results indicate clearly that the autoimmune disease-related peptide 17′ does not significantly differ from the negative control peptide whilst peptides GHB-H1 and GHB-G1 show absorbances above 0.50 and almost 1.50 respectively at melanoma sera pool dilution 1:50. GHB-G1 gave a signal of 1.50 even at a melanoma: sera pool dilution 1:200.

### Example 10

### Analysis of Serum Samples from Melanoma Patients

For analysis of serum samples from melanoma patients, Nunc Polysorp Immobilizer Amino plates were used. Compared with Maxisorp plates (Nunc), Polysorp plates showed 25% higher absorbance in melanoma sera and 10% lower absorbance in negative sera. The optimised ELISA system was tested by using 31 serum samples from melanoma patients. 16 serum samples from healthy individuals served as controls to establish a negative treshold, as calculated by the average absorbance plus three standard deviations. A value of 0.39 or above is defined as positive. The results for the G1 epitope are shown in FIG. 6 indicating clearly that 15 out of the 31 melanoma serum samples reacted positive whilst 16 melanoma sera did not recognise the G1 epitope.

### REFERENCES

Venter et al., Science 291:1304-51, 2001
Lower et al., Proc Natl Acad Sci USA. 93(11):5177-84, 1996
Barnstable et al., Cell 14(1):9-20, 1978
Bronson et al., J. Natl. Cancer Inst. 60, 1305-1308, 1978
Lower et al., J. Gen. Virol. 65, 887-898, 1984
Turner et al., Curr. Biol. 11, 1531-1535, 2001
Mayer et al., Nat Genet. 21(3):257-8, 1999
Muster et al., Cancer Res. 15; 63(24):8735-41, 2003
Kleiman et al., Int J. Cancer. 110(3):459-61, 2004
Kirkwood and Agarwala, Principles and Practice of Oncology 7: 1-16, 1993
Herve et al., Clin Exp Immunol. 128(1):75-82, 2002
Goedert et al., Cancer Epidemiol Biomarkers Prey. 8(4 Pt 1):293-6, 1999
Boller et al., J. Virol. 71(6):4581-8, 1997
Reineke et al., J Immunol Methods 267:37, 2002
Sauter et al., J. Virol. 69(1):414-21, 1995
Singh et al., Nature Biotech. 17: 1075-1081, 1999
Depil et al., Leukemia. 16(2):254-9, 2002
Moles et al., Virus Res. 94(2):97-101, 2002
Balch et al., J Clin Oncol. 19(16):3635-48, 2001
King et al., Transcript of “Conference on Scientific Issues Related to Potential Allergenicity in Transgenic Food Crops,” Apr. 18-19, 1994.

1. An isolated antigenic fragment of the env-protein of a melanoma-associated endogenous retrovirus MERV comprising at least 6 continuous amino acids of the sequence of YQRSLKFRPKGKPCPKE (SEQ ID NO: 7) or a mimotope thereof.

2. The isolated antigenic fragment of claim 1, comprising at least 8 continuous amino acids of the amino acid sequence of SEQ ID NO: 7 or a mimotope thereof.

3. The isolated antigenic fragment of claim 1, comprising between 8 to 15 continuous amino acids of the amino acid sequence of SEQ ID NO: 7 or a mimotope thereof.

4. The isolated antigenic fragment of claim 1, comprising the amino acid sequence of SEQ ID NO: 7 or a mimotope thereof.

5. The isolated antigenic fragment of claim 4, comprising the amino acid sequence of SEQ ID NO: 7.

6. The isolated antigenic fragment of claim 4, comprising the amino acid sequence of a mimotope of the sequence of SEQ ID NO: 7.

7. The isolated antigenic fragment of claim 1, comprising any one of the amino acid sequences of YQRSLK (SEQ ID NO:75), QRSLKFRPKGKP (SEQ ID NO:76), RSLKFRPKGK (SEQ ID NO:77), SLKFRPKGKPCP (SEQ ID NO:78), FRPKGKPCP (SEQ ID NO:79), KGKPCPK (SEQ ID NO:80), GKPCPKE (SEQ ID NO:81), KGKPCPKEIPKESK (SEQ ID NO:84), SYQRSLKFRPK (SEQ ID NO:86), YQRSLKFRP (SEQ ID NO:87), RSLKFRP (SEQ ID NO:88), KGKPCPKEI (SEQ ID NO:89), FRPKGKPCPKEIP (SEQ ID NO:90), or GKPCPKEIPK (SEQ ID NO:91).

8. The isolated antigenic fragment of claim 1, further comprising a covalently bound biotin.

9. An isolated antibody directed against an antigenic fragment of claim 1.

10. A method of detecting an anti-MERV-antibody, if any, in a sample comprising:
contacting a sample with an antigenic fragment of claim 1, leading to antibody-antigen binding between an anti-MERV antibody, if any, in the sample and the antigenic fragment; and
detecting any anti-MERV antibody in the sample by detecting the binding between the anti-MERV antibody and the antigenic fragment.

11. The method of claim 10, further comprising quantifying the anti-MERV antibody in the sample.

12. The method of claim 11, wherein the anti-MERV antibody is quantified by either determining the amount of antibody-bound antigenic fragment, or the amount of antigenic fragment-bound antibody, or the amount of antibody-free antigenic fragment, or the amount of antigenic fragment-free antibody.

13. The method of claim 10, wherein the antigenic fragment is immobilized on a surface.

14. The method of claim 10, wherein the amount of antibody-free antigenic fragment is detected by at least one additional secondary antibody, which creates a detectable marker signal.

15. The method of claim 10, wherein the binding between the anti-MERV antibody and the antigenic fragment is detected using an enzyme-linked immunosorbent assay.

16. A method of detecting a MERV protein or MERV protein fragment in a sample comprising:
contacting a sample with an antibody or antibody fragment directed against an antigenic fragment of claim 1, leading to antibody-antigen binding between the anti-body and a MERV protein or MERV protein fragment, if any, in the sample; and
detecting any MERV protein or MERV protein fragment in the sample by detecting the binding between the antibody and the MERV protein or MERV protein fragment

17. The method of claim 16, further comprising quantifying the MERV protein or MERV protein fragment in the sample.

18. The method of claim 17, wherein the antibody-bound MERV protein or MERV protein fragment is quatified by either determining either an amount of MERV protein- or MERV protein fragment-bound antibody, if any, or an amount of antibody-free MERV protein or MERV protein fragment, if any, or an amount of MERV protein- or MERV protein fragment-free antibody if any.

19. The method of claim 16, comprising using an antigenic fragment of claim 1 as a competitive antigen.

20. The method of claim 19, wherein the competitive antigen is immobilized to a surface.
